(19) United States
(12) Patent Application Publication
(10) Pub. No.: US 2023/0187059 A1
(43) Pub. Date: Jun. 15, 2023

(54) AUTOMATED TICKET ATTACHMENT CREATION

(71) Applicant: KONINKLIJKE PHILIPS N.V., Eindhoven (NL)

(72) Inventors: Joachim Dieter SCHMIDT, HAMBURG (DE); Thomas Erik AMTHOR, HAMBURG (DE)

(21) Appl. No.: 18/076,452

(22) Filed: Dec. 7, 2022

Related U.S. Application Data
(60) Provisional application No. 63/289,712, filed on Dec. 15, 2021.

(51) Int. Cl. G16H 40/40; G06Q 30/015

(52) U.S. Cl. CPC G16H 40/40; G06Q 30/015

(57) ABSTRACT

A non-transitory computer readable medium (26s) stores instructions executable by at least one electronic processor (14s) to perform an automated ticketing method (100) including receiving a support request ticket (40) for assistance with a medical device (2), the support request ticket being entered by a requestor via a ticket entry user interface (UI) (42) of an automated ticket management system; analyzing the support request ticket to identify ticket enhancement information not included in the support request ticket; retrieving the ticket enhancement information from one or more databases (46, 48); adding the ticket enhancement information to the support request ticket to generate an enhanced support request ticket (50); and transmitting one of the support request ticket or the enhanced support request ticket to a ticket recipient electronic processing device (12) operable by a ticket recipient.

### CROSS REFERENCE TO RELATED APPLICATIONS

This application claims the benefit of U.S. Provisional Pat. Application No. 63/289,712 filed Dec. 15, 2021. This application is hereby incorporated by reference herein.

The following relates generally to the support request ticketing arts, the imaging arts, remote imaging assistance arts, medical device maintenance arts, medical imaging device maintenance arts, assistance ticket generation arts, and related arts.

### BACKGROUND

Communication inside and outside of healthcare providers may include channels like chatting or screen sharing with local staff or remote experts to discuss challenging clinical situations, equipment matters, and so forth, reporting medical equipment malfunctions, and so forth. The management of these collaboration type interactions, equipment issue reporting, and so forth among users may be based on tickets, and an electronic ticket management system can be used similar to such systems that are well known from customer service provided by central help desks or information technology (IT) help desks. As used herein, a “support request ticket” or simply “ticket” refers to a request for support on a particular problem that emerged during normal operations of a business unit. It can be seen as a request to perform a task outside the normal way of working. Typically, a ticket has a sender and a recipient. The latter is supposed to provide support to the sender. The type of task that is initiated by a ticket can be a collaboration between the sender and the recipient or a task that is solely performed by the recipient (for example, an equipment repair task performed by a technician).

As used herein, a ticket management system refers to software used to register, organize, prioritize, and resolve support tickets. These systems often encompass resource allocation, time accounting, priority management, and oversight workflow in addition to implementing a centralized ticket registry. A ticket management system can significantly moderate the interrupting nature of ad-hoc collaboration like phone calls or in person inquiries as it is of asynchronous nature thus allowing the recipient of a ticket to pick a convenient time slot for the interaction. Beside the appropriate routing of tickets, information that is attached to a ticket can improve the efficiency of ticket processing significantly. If information providing the context of a ticket is attached to a ticket, time can be saved when a ticket is dealt with. The better a ticket context is in line with the topic the ticket is about, the less time must be spent to get the context when starting a collaboration or other ticket resolution.

Ticket systems in healthcare differ from ticket systems that are used by help desk services. While the latter provide a central handling of tickets by several agents that are often preselected depending on the topic of the ticket, in healthcare the recipients of tickets are distributed and handle often tasks of different nature. If the recipient picks up a ticket, e.g. one that requests a collaboration on a medically related problem, the recipient needs to switch work context. For example, a radiologist, who receives a ticket from a technologist about changing an imaging protocol, needs to get familiar with this patient case in a most effective way. Existing electronic support request ticketing systems typically provide limited contextual information.

The following discloses certain improvements to overcome these problems and others.

### SUMMARY

In one aspect, a non-transitory computer readable medium stores instructions executable by at least one electronic processor to perform an automated ticketing method including receiving a support request ticket for assistance with a medical device, the support request ticket being entered by a requestor via a ticket entry user interface (UI) of an automated ticket management system; analyzing the support request ticket to identify ticket enhancement information not included in the support request ticket; retrieving the ticket enhancement information from one or more databases; adding the ticket enhancement information to the support request ticket to generate an enhanced support request ticket; and transmitting one of the support request ticket or the enhanced support request ticket to a ticket recipient electronic processing device operable by a ticket recipient.

In another aspect, a non-transitory computer readable medium stores instructions executable by at least one electronic processor to perform an automated ticketing method between an operator of a medical device and a ticket recipient being requested to assist the operator. The method includes analyzing a support request ticket to identify ticket enhancement information not included in the support request ticket, the support request ticket including a request for assistance with a medical device and being entered by a requestor via a ticket entry UI of an automated ticket management system; retrieving the ticket enhancement information from one or more databases; adding the ticket enhancement information to the support request ticket to generate an enhanced support request ticket; transmitting one of the support request ticket or the enhanced support request ticket to a ticket recipient electronic processing device operable by a ticket recipient; displaying the enhanced support request ticket via the ticket entry UI; and receiving, from the requestor, via the ticket entry UI, a user input indicative of one of an acceptance of the enhanced support request ticket or a rejection of the enhanced support request ticket.

In another aspect, an automated ticketing method between a local operator performing a servicing session for a medical device and a remote expert assisting the local operator including analyzing a ticket for assistance submitted by the local operator to the remote expert with a machine learning (ML) component to identify ticket enhancement information not included in the ticket; retrieving the ticket enhancement information from one or more databases; filling in one or more fields of the ticket with the retrieved ticket enhancement information to generate an enhanced ticket; and transmitting the enhanced ticket to an electronic processing device operable by the remote expert.

One advantage resides in generating request tickets from a requestor to a ticket recipient.

Another advantage resides in automatically selecting data that is required for an efficient ticket processing for the ticket recipient.

Another advantage resides in using a machine learning (ML) component to select information to be provided in a ticket.

Another advantage resides in reducing an amount of information that needs to be added to a ticket.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 diagrammatically shows an illustrative ticket management apparatus in accordance with the present disclosure.

FIG. 2 shows an example flow chart of automated ticket management operations suitably performed by the apparatus of FIG. 1.

FIGS. 3 and 4 show example tickets generated by the apparatus of FIG. 1.

FIGS. 5-7 show example flow charts of operations suitably performed by the apparatus of FIG. 1.

FIG. 8 shows another example of the apparatus of FIG. 1.

### DETAILED DESCRIPTION

There is interest in developing an automated ticketing system for communicating non-urgent requests between imaging technicians and radiologists or other persons involved in radiology operations. In a medical context, the recipient may be a skilled professional such as a radiologist, who handles a wide range of different tasks. For example, a radiologist is a medical doctor with an expansive range of responsibilities in many organizations. A radiologist may assist imaging technologists in setting up imaging examinations, provide rapid image quality review during an imaging examination, perform readings of completed imaging examinations and draft medical reports thereon (referred to as a “radiology reports” in some settings), provide professional advice on matters such as imaging equipment acquisition, and so forth. When handling an electronic support request ticket, the radiologist needs to get quickly become familiar with the patient case, imaging device, or other information related to the topic of the ticket in a most effective way and preferably with a minimum of additional information collection. On the other hand, the information that is collected should be sufficient to handle the topic, and should be as concise as possible to save time and effort. Providing too much data can be counter-productive as the recipient needs to manually extract the information that is relevant. Furthermore, while the recipient’s (e.g. radiologist’s) time is valuable, the sender’s time is also valuable. Requiring the sender to collect the essential information manually and attach it to the ticket just shifts burden from one party to the other.

Resolution of a radiology ticket is likely to involve patient records from the Electronic Medical Record (EMR), imaging examination information from a Radiology Information System (RIS), and possibly other data from other sources such as equipment maintenance records. Disclosed herein are embodiments of a sub-system operating in conjunction with an automated ticketing system for automatically collecting these data and attaching same to the ticket.

To do this efficiently, in some embodiments the various data sources are structured in accordance with Domain Object Model (DOM) formatting. Additionally or alternatively, natural language processing (NLP) can be used to extract information from unstructured databases, or from free-form data fields of a DOM.

A machine learning (ML) component is trained to analyze the basic ticket information (sender, recipient, priority, problem description, et cetera) and to identify fields of the DOM for attachment to the ticket. The ML component may, for example, be an artificial neural network (ANN). The content of the identified fields is retrieved from the relevant database(s) and attached to the ticket.

The resulting enhanced ticket is proposed to the sender, who can either accept or reject the additions. In case of rejection, the original ticket is sent to the recipient. In case of acceptance, the enhanced ticket is sent to the recipient.

The recipient then retrieves and process the ticket. The system in some embodiments tracks which enhancement information (if any) is or is not used, and optionally also tracks any additional information that was not attached to the ticket that the recipient retrieves while processing the ticket. Notably, the latter tracking of additional information can be done even if the original ticket is sent to the recipient. The tracking is suitably implemented by adding data retrieval monitoring to the automatic ticketing system, and the result is information about which additional information was used, which was not, and what additional information should have been attached to the ticket. This constitutes a training ticket, that can optionally be fed back to perform further training of the ML component. Optionally, the recipient can choose whether to submit the training ticket for use in training.

In some embodiments, a reliability score is automatically assigned to the enhanced ticket. If this score is too low, then the sender is not given the option to review and send the enhanced ticket. This can be done for the initial iterations, for example, to avoid annoying the sender by suggesting an enhanced ticket that is likely to be wrong. In addition, if the score is too low, no automatic attachment of the score to the ticket is created. Instead, the sender would need to attach all required information manually.

In illustrative embodiments, the disclosed automated ticket management system is deployed in a radiology context, and in conjunction with an on-call remote expert assistance system. However, this is merely an illustrative embodiment, and in other contemplated embodiments the automated ticket management system may be deployed in other medical domains besides radiology, and even more broadly in non-medical domains, and with or without (or independently of) an on-call remote expert assistance system.

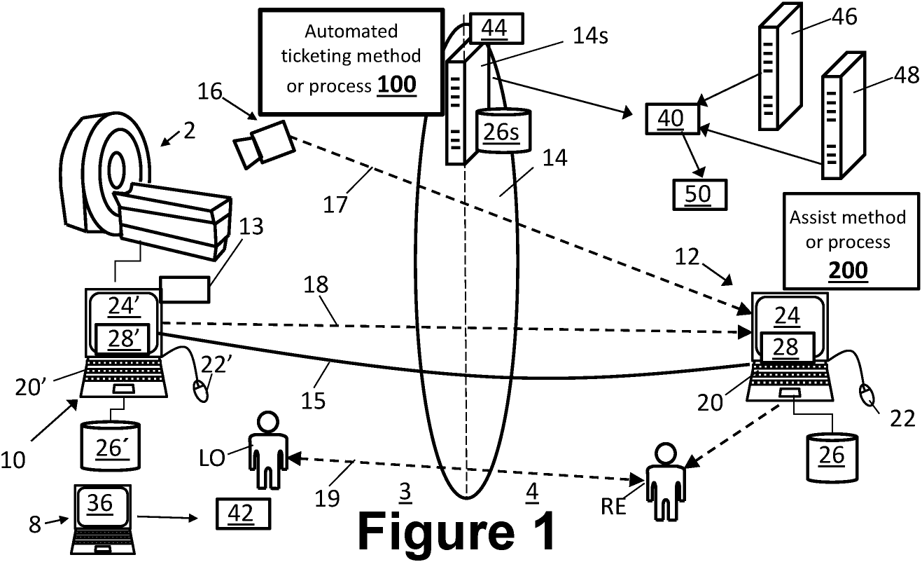

With reference to FIG. 1, an apparatus for providing assistance from a ticket receiver R, such as a remote medical imaging expert RE (or supertech such as an experienced technologist), a radiologist, and so forth to a ticket sender S, such as a local radiologist technician or local technician operator LO is shown. Such a system is also referred to herein as a radiology operations command center (ROCC). As shown in FIG. 1, the ticket sender S can be the local operator LO, who operates a medical imaging device (also referred to as an image acquisition device, imaging device, and so forth) 2, is located in a medical imaging device bay 3, and the ticket receiver R is a remote expert RE is disposed in a remote service location or center 4. It should be noted that the remote expert RE may not necessarily directly operate the medical imaging device 2, but rather provides assistance to the local operator LO in the form of advice, guidance, instructions, or the like. The remote location 4 can be a remote service center, a radiologist’s office, a radiology department, and so forth. The remote location 4 may be in the same building as the medical imaging device bay 3 (this may, for example, in the case of a remote expert RE who is a radiologist tasked with peri-examination image review), or the remote service center 4 and the medical imaging device bay 3 may be in different buildings, and indeed may be located in different cities, different countries, and/or different continents. In general, the remote location 4 is remote from the imaging device bay 3 in the sense that the remote expert RE cannot directly visually observe the imaging device 2 in the imaging device bay 3 (hence optionally providing a video feed as described further herein).

The image acquisition device 2 can be a Magnetic Resonance (MR) image acquisition device, a Computed Tomography (CT) image acquisition device; a positron emission tomography (PET) image acquisition device; a single photon emission computed tomography (SPECT) image acquisition device; an X-ray image acquisition device; an ultrasound (US) image acquisition device; or a medical imaging device of another modality. The imaging device 2 may also be a hybrid imaging device such as a PET/CT or SPECT/CT imaging system. While a single image acquisition device 2 is shown by way of illustration in FIG. 1, more typically a medical imaging laboratory will have multiple image acquisition devices, which may be of the same and/or different imaging modalities. The remote service center 4 may provide service to multiple hospitals. The local operator LO controls the medical imaging device 2 via an imaging device controller 10. The remote expert RE is stationed at a remote workstation 12 (or, more generally, an electronic controller 12).

The imaging device controller 10 includes an electronic processor 20′, at least one user input device such as a mouse 22′, a keyboard, and/or so forth, and a display device 24′. The imaging device controller 10 presents a device controller graphical user interface (GUI) 28′ on the display 24′ of the imaging device controller 10, via which the local operator LO accesses device controller GUI screens for entering the imaging examination information such as the name of the local operator LO, the name of the patient and other relevant patient information (e.g. gender, age, etc.) and for controlling the (typically robotic) patient support to load the patient into the bore or imaging examination region of the imaging device 2, selecting and configuring the imaging sequence(s) to be performed, acquiring preview scans to verify positioning of the patient, executing the selected and configured imaging sequences to acquire clinical images, display the acquired clinical images for review, and ultimately store the final clinical images to a Picture Archiving and Communication System (PACS) or other imaging examinations database. In addition, while FIG. 1 shows a single medical imaging device bay 3, it will be appreciated that the remote service center 4 (and more particularly the remote workstation 12) is typically in communication with multiple medical bays via a communication link 14, which typically comprises the Internet augmented by local area networks at the remote operator RE and local operator LO ends for electronic data communications.

As diagrammatically shown in FIG. 1, in some embodiments, a camera 16 (e.g., a video camera) is arranged to acquire a video stream 17 of a portion of the medical imaging device bay 3 that includes at least the area of the imaging device 2 where the local operator LO interacts with the patient, and optionally may further include the imaging device controller 10. The video stream 17 is sent to the remote workstation 12 via the communication link 14, e.g., as a streaming video feed received via a secure Internet link. In some examples, as shown in FIG. 1, the camera 16 can be affixed to a wall of ceiling of the medical facility with a field of view to include the area of the imaging device 2 where the local operator LO interacts with the patient, and optionally may further include the imaging device controller 10. In other examples, the camera 16 can be disposed within an imaging bore (not shown) of the imaging device 2.

In other embodiments, the live video feed 17 of the display 24′ of the imaging device controller 10 is, in the illustrative embodiment, provided by a video cable splitter 15 (e.g., a DVI splitter, a HDMI splitter, and so forth). In other embodiments, the live video feed 17 may be provided by a video cable connecting an auxiliary video output (e.g. aux vid out) port of the imaging device controller 10 to the remote workstation 12 operated by the remote expert RE. Alternatively, a screen mirroring data stream 18 is generated by screen sharing software 13 running on the imaging device controller 10 which captures a real-time copy of the display 24′ of the imaging device controller 10, and this copy is sent from the imaging device controller 10 to the remote workstation 12. Other approaches besides the illustrative video cable splitter 15 or screen sharing software 13 are contemplated for capturing a real-time copy of the display 24′ of the imaging device controller 10 which is then sent to the workstation 12 of the remote expert RE. While in an ROCC this real-time copy of the display 24′ of the imaging device controller 10 is used to provide status information to the remote expert RE for use in assisting the local operator LO, in embodiments disclosed herein the real-time copy of the display 24′ of the imaging device controller 10 is also leveraged (optionally along with other available information) to determine one or more performance metrics of the local operator LO.

The communication link 14 also provides a natural language communication pathway 19 for verbal and/or textual communication between the local operator LO and the remote expert RE, in order to enable the latter to assist the former in performing the imaging examination. For example, the natural language communication link 19 may be a Voice-Over-Internet-Protocol (VOIP) telephonic connection, a videoconferencing service, an online video chat link, a computerized instant messaging service, or so forth. Alternatively, the natural language communication pathway 19 may be provided by a dedicated communication link that is separate from the communication link 14 providing the data communications 17, 18, e.g., the natural language communication pathway 19 may be provided via a landline telephone. In another example, the natural language communication pathway 19 may be provided via an ROCC device 8, such as a mobile device (e.g., a tablet computer or a smartphone), or can be a wearable device worn by the local operator LO, such as an augmented reality (AR) display device (e.g., AR goggles), a projector device, a heads-up display (HDD) device, etc., each of which having a display device 36. For example, an “app” can run on the ROCC device 8 (operable by the local operator LO) and the remote workstation 12 (operable by the remote expert RE) to allow communication (e.g., audio chats, video chats, and so forth) between the local operator and the remote expert.

FIG. 1 also shows, in the remote service center 4 including the remote workstation 12, such as an electronic processing device, a workstation computer, or more generally a computer, which is operatively connected to receive and present the video 17 of the medical imaging device bay 3 from the camera 16 and to present the screen mirroring data stream 18 as a mirrored screen. Additionally or alternatively, the remote workstation 12 can be embodied as a server computer or a plurality of server computers, e.g., interconnected to form a server cluster, cloud computing resource, or so forth. The workstation 12 includes typical components, such as an electronic processor 20 (e.g., a microprocessor), at least one user input device (e.g., a mouse, a keyboard, a trackball, and/or the like) 22, and at least one display device 24 (e.g., an LCD display, plasma display, cathode ray tube display, and/or so forth). In some embodiments, the display device 24 can be a separate component from the workstation 12. The display device 24 may also comprise two or more display devices, e.g., one display presenting the video 17 and the other display presenting the shared screen (i.e., display 24′) of the imaging device controller 10 generated from the screen mirroring data stream 18. Alternatively, the video and the shared screen may be presented on a single display in respective windows. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the workstation 12, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the remote operator display device 24.

The medical imaging device controller 10 in the medical imaging device bay 3 also includes similar components as the remote workstation 12 disposed in the remote service center 4. Except as otherwise indicated herein, features of the medical imaging device controller 10 disposed in the medical imaging device bay 3 similar to those of the remote workstation 12 disposed in the remote service center 4 have a common reference number followed by a “prime” symbol (e.g., processor 20′, display 24′, GUI 28′) as already described. In particular, the medical imaging device controller 10 is configured to display the imaging device controller GUI 28′ on a display device or controller display 24′ that presents information pertaining to the control of the medical imaging device 2 as already described, such as imaging acquisition monitoring information, presentation of acquired medical images, and so forth. It will be appreciated that the real-time copy of the display 24′ of the controller 10 provided by the video cable splitter 15 or the screen mirroring data stream 18 carries the content presented on the display device 24′ of the medical imaging device controller 10. The communication link 14 allows for screen sharing from the display device 24′ in the medical imaging device bay 3 to the display device 24 in the remote service center 4. The GUI 28′ includes one or more dialog screens, including, for example, an examination/scan selection dialog screen, a scan settings dialog screen, an acquisition monitoring dialog screen, among others. The GUI 28′ can be included in the video feed 17 or provided by the video cable splitter 15 or by the mirroring data stream 17′ and displayed on the remote workstation display 24 at the remote location 4.

Where there is a multiplicity of local operators LO and multiplicity of remote operators RO, the disclosed communication link 14 may suitably include a server computer 14s (or a cluster of servers, cloud computing resource comprising servers, or so forth) which is programmed to establish connections between selected local operator LO/remote expert RE pairs. For example, if the server computer 14s is Internet-based, then connecting a specific selected local operator LO/remote expert RE pair can be done using Internet Protocol (IP) addresses of the various components 16, 10, 12.

As an illustrative example of operation of the ROCC, the workstation 12 in the remote location 4 is programmed to receive at least one of: (i) the video 17 from the video camera 16 of the medical imaging device 2 located in the medical imaging device bay 3; and/or (ii) the screen sharing 18 from the screen sharing software 13; and/or (iii) the video 17 tapped by the video cable splitter 15. The video feed 17 and/or the screen sharing 18 can be displayed at the remote workstation display 24, typically in separate windows of the GUI 28. The video feed 17 and/or the screen sharing 18 can be screen-scraped to determine information related to the medical imaging examination (e.g., modality, vendor, anatomy to be imaged, cause of issue to be resolved, and so forth). In particular, the GUI 28 presented on the display 24 of the remote workstation 12 preferably includes a window presenting the video 17, and a window presenting the mirrored screen of the medical imaging device controller 10 constructed from the screen mirroring data stream 18, and status information on the medical imaging examination that is maintained at least in part using the screen-scraped information. This allows the remote operator RE to be aware of the content of the display of the medical imaging device controller 10 (via the shared screen) and also to be aware of the physical situation, e.g., position of the patient in the medical imaging device 2 (via the video 17), and to additionally be aware of the status of the imaging examination as summarized by the status information. During an imaging procedure, the natural language communication pathway 19 is suitably used to allow the local operator LO and the remote operator RE to discuss the procedure and in particular to allow the remote operator to provide advice to the local operator.

The ROCC device 8, the remote workstation 12, and the server computer 14s can further implement an automated ticket management system for the local operator LO (or, alternatively, a requestor) to generate a support request ticket 40 for the remote expert RE (or, alternatively, a ticket recipient, such as the remote expert RE, an administrator, an IT department, and so forth ). To do so, a ticket entry UI 42 can be implemented on the display device 36 of the ROCC device 8. The requestor can then use the ticket entry UI 42 to generate the support request ticket 40. The support request ticket 40 is then transmitted to the server computer 14s, which includes a machine-learning component 44 (i.e., a neural network, such as an artificial neural network (ANN)) to process the support request ticket 40. For example, the ML component can retrieve data from one or more databases, such as patient data from an electronic medical record (EMR) database 46, data about a medical examination performed for the patient from a radiology information system (RIS) database 48, and so forth. Data from the databases 46, 48 can be used to augment the support request ticket 40 to generate an enhanced support request ticket 50, which is then transmitted to the remote workstation 12.

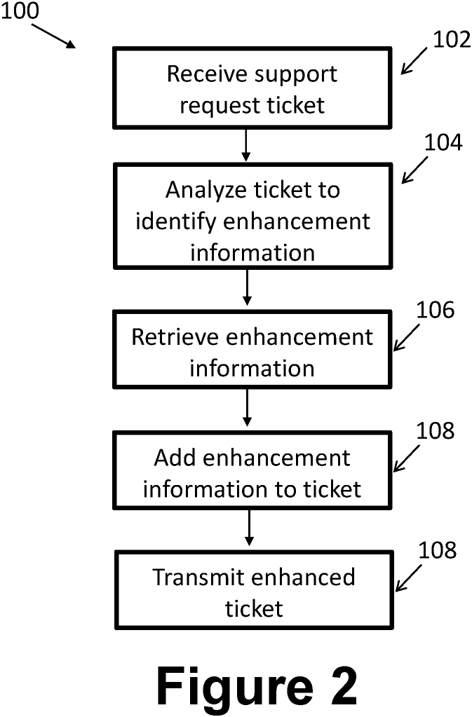

Furthermore, as disclosed herein the server 14s performs an automated ticketing method or process 100 for generating and transmitting the enhanced support request ticket 50 to the ticket recipient. With reference to FIG. 2, and with continuing reference to FIG. 1, an illustrative embodiment of the ticketing method 100 is diagrammatically shown as a flowchart. At an operation 102, the support request ticket 40 for assistance with the medical device 2 is received at the server computer 14s from the ROCC device 8. The support request ticket 40 is entered by the requestor or ticket sender S via the ticket entry UI 42 on the ROCC device 8.

At an operation 104, the support request ticket 40 is analyzed to identify ticket enhancement information not included in the support request ticket. To do so, the support request ticket 40 is input to the ANN 44 of the server computer 14s that has been pre-trained (and/or is being dynamically trained) to identify the ticket enhancement information.

At an operation 106, the ticket enhancement information is retrieved from one or more databases. In one example, the ticket enhancement information can include patient data retrieved from the EMR database 46. In another (non-mutually exclusive) example, the ticket enhancement information can include data about the medical examination performed for the patient from the RIS database 48. In some embodiments, the retrieved the ticket enhancement information comprises a domain object model (DOM) formatting information.

At an operation 108, the retrieved ticket enhancement information is added to the support request ticket 40 to generate the enhanced support request ticket 50. This can be performed by adding the retrieved ticket enhancement information to fields in the support ticket request 40, or attached to the support ticket request 40. In some embodiments, a reliability score can be assigned for the enhanced support request ticket 50.

At an operation 110, either the support request ticket 40 or the enhanced support request ticket 50 is transmitted to the ticket recipient electronic processing device (i.e., the remote workstation 12) operable by a ticket recipient such as the ticket receiver R. In some embodiments, the enhanced support request ticket 50 can then be displayed on the ticket entry UI 42 on the ROCC device 8. The requestor can then input (e.g., via a finger swipe, a finger tap , and so forth) a user input indicative of one of an acceptance of the enhanced support request ticket 50 or a rejection of the enhanced support request ticket 50. The enhanced support request ticket 50 is transmitted to the ticket recipient electronic processing device 12 in response to receiving an acceptance (or an indication of a rejection) of the enhanced support request ticket 50.

In some examples, when the enhanced support request ticket 50 is transmitted to the ticket receiver R at the remote workstation 12, the remote workstation 12 can track which ticket enhancement information are used by the ticket recipient to assist with the medical device 2, and training of the ANN 44 can be updated based on which ticket enhancement information are used by the ticket recipient to assist with the medical device 2. In another example, the remote workstation 12 can track other information that is not included in the ticket enhancement information that is used by the ticket receiver R to assist with the medical device 2, and the training of the ANN 44 can be updated based on the other information. In some examples, since the remote expert RE may be reviewing the patient imaging real time, the ANN 44 can continue to run in the background and continue to enhance to the ticket information based on the review aspects of the remote expert RE. To do so, the remote expert RE can provide one or more user inputs via the at least one user input device 22 of the remote workstation 12, and the ANN 44 is configured to apply these inputs to the support request ticket 40 to generate the enhanced support request ticket 50.

The method 100 can be performed in a medical context, in particular a medical imaging procedure context. For example, the ticket enhancement information can be determined by one or more of an imaging protocol, a modality of the medical imaging device 2, a status of the medical imaging device 2, a clinical pathway of the medical imaging procedure and so forth. In another example, the ticket enhancement information can be determined by one or more of information about the sender, wherein such sender information includes at least one of experience level, job description, general training experience, and specific training experience on the particular imaging protocol, imaging modality/brand, or clinical pathway.

The automated ticket management system of FIG. 1 is implemented in conjunction with the ROCC, thereby providing an efficient way for the local operator LO (or, alternatively, a requestor) to generate a support request ticket 40 for the remote expert RE (or, alternatively, a ticket recipient). The automated ticket management system is useful in conjunction with the ROCC as it provides a mechanism for the local operator LO and the remote expert RE to efficiently communicate in an asynchronous manner to arrange effective collaborations. However, as previously noted, the automated ticket management system can be usefully deployed in other medical collaborative contexts, such as to enable a physician to request assistance of a medical specialist (e.g., a radiologist, histopathologist, orthopedist, nutritionist, or so forth). In other embodiments, the automated ticket management system may be usefully deployed to enable a medical professional to request technical assistance respecting a piece of medical equipment from a staff technician, IT professional, or the like. Still further, the automated ticket management system may find application in fields outside of medicine.

In the following, some further illustrative examples of embodiments of automated ticket management systems are described. In the associated flowchart and diagrammatic domain object model (DOM) drawings, solid connectors indicate data transfer or flow pathways or the like between blocks of the flowchart or DOM representation, while dashed connectors associate such blocks to comments which are indicated by a turned upper-right corner of the comment box.

### EXAMPLE

The disclosed system is used in routine healthcare operations that are based on control and tracking of the tasks of a pre-defined process (i.e., process model). The disclosed workflow management system provides the process control and tracking data. If an ad-hoc task needs to be performed while handling a task that is part of a process, the disclosed workflow management system takes care of adding all data that is required by the recipient to be able to process the task. The trigger of an ad-hoc task (as used herein, refers to work items that users or algorithms (i.e., AI-systems) can create extemporaneously that are not part of a modelled process flow. Ad hoc tasks can be performed or scheduled within the context of a process or outside the context of a process) can be the subject of a ticket (i.e., the support ticket request 40) that contains all necessary context data. The recipient of the support ticket request 40 gets, when processing the support ticket request 40, all required information without any additional actions.

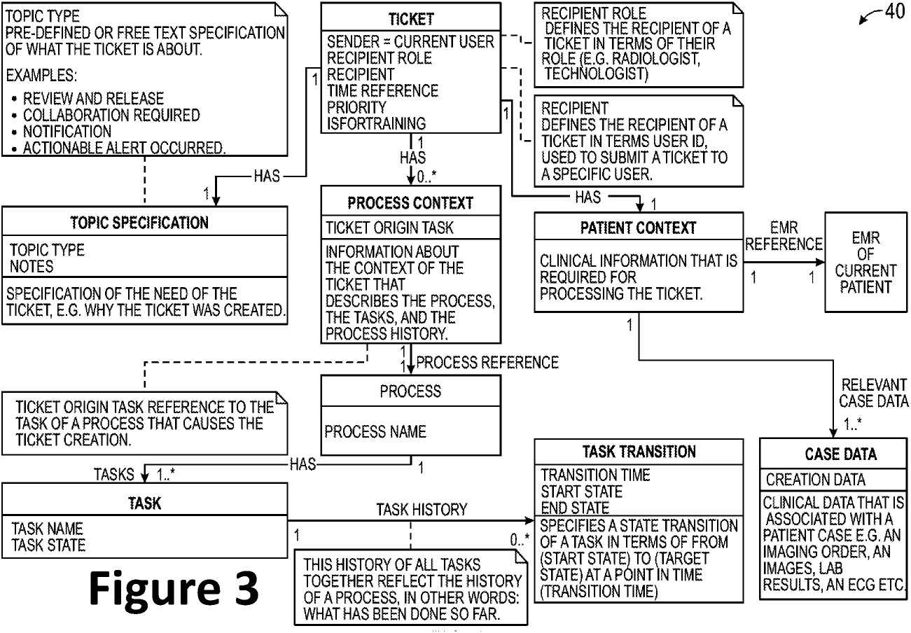

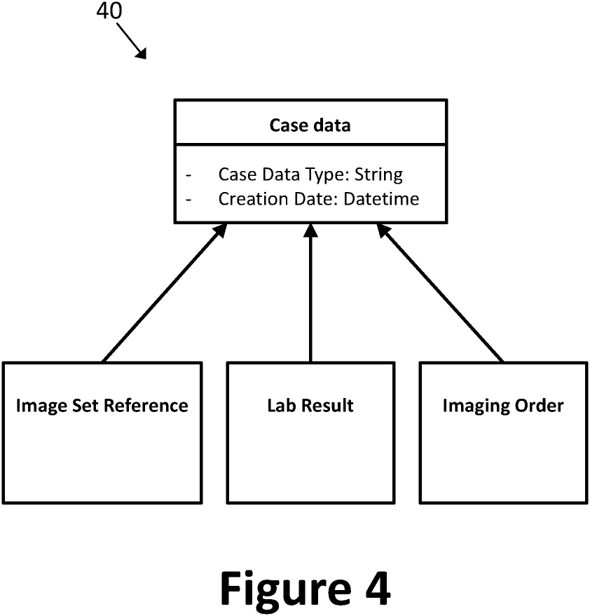

FIGS. 3 and 4 show examples of the support ticket request 40. FIG. 3 shows an example of a domain object model (DOM) of information to be included in the support ticket request 40, and FIG. 4 shows an example of a DOM for the case data of the support ticket request 40. The DOM specifies classes of tasks to be performed in the support ticket request 40. The support ticket request 40 can be linked to the requestor, can specify the ticket recipient, can have a time stamp, and can include an urgency level. The support ticket request 40 can include a topic, a process context, and/or a patient context for assistance by the ticket recipient. The process context comprises a compact representation of the process state and the task states at the time when the support ticket request 40 was created. It states the task, which is associated with a ticket, and gives information about the current state of other tasks including their state transition changes over time. This task history serves as input to the ANN 44 for the determination of the patient context and the selection of relevant case data. The patient context provides the information for the ticket recipient that is needed to process the support ticket request 40.

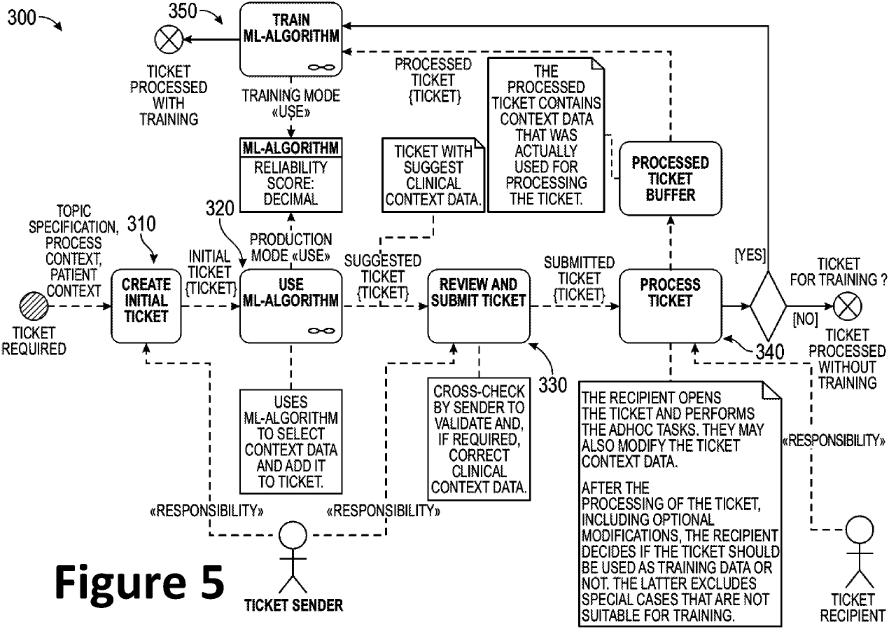

The ANN 44 is configured (e.g., trained on suitable training data) to identify items of the patient context that have a highest relevance for the ticket recipient. The items can include, for example, patient demographic details, patient referral status (i.e., inpatient/outpatient/emergency/etc.), patient physical or mental condition, patient medical history events, patient medication details, information about other ongoing medical procedures, previously-acquired or current medical images, previous radiologic reports, previous tickets concerning the patient, contact details of referring physician or any other previously involved medical professional, comments about the case provided by other medical professionals, lab results, and so forth. The identification of relevant case data items is performed by the ANN 44 by assigning relevance scores to each possible case data item, based on a set of features provided as input to the ANN 44. These features can include, for example, ticket topic, meta data from previous images, keywords, point in time of latest patient/case details, and process context (including resource identifiers and experience levels of human resources). It should be noted that the illustrative DOM representations of FIGS. 3 and 4 are illustrative examples, and numerous variants are contemplated based on the type of ticket and other factors. The illustrative DOM’s of FIGS. 4 and 5 are suitable examples for radiology support request tickets.

FIG. 5 shows an automated ticketing method or process method 300 which can suitably be substituted for the automated ticketing method or process 100 of FIG. 1. At an operation 310 an initial ticket (i.e., the support ticket request 40) is created. To do so, the initial ticket includes a topic, a process context, and/or a patient context for assistance by the ticket recipient. At an operation 320, the ML algorithm (i.e., the ANN 44) is used to analyze the initial ticket.

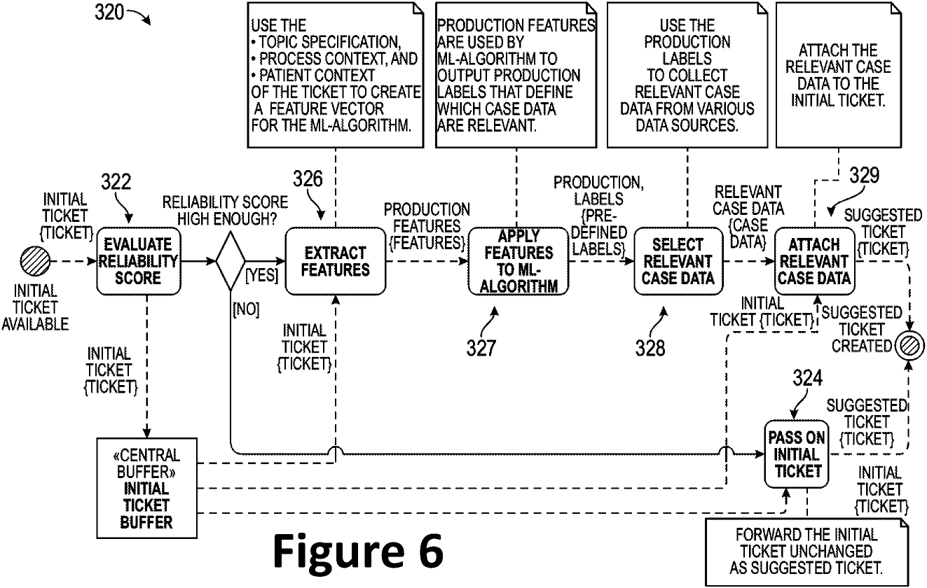

FIG. 6 shows a detailed example of the operation 320. At an operation 322, a reliability score of the initial ticket determined by the ML algorithm is evaluated. In case the score is too low, the initial ticket is passed on and the algorithm is not used (see operation 324). If the score is high enough, the data of the initial ticket is used to generate the input features for the ML-algorithm (see operation 326). These features are applied into the ML algorithm to obtain the predefined labels with their relevance scores (see operation 327). The labels are associated with context data and the scores decide if the data is required. The relevance score is either 0 (“data not needed”), or 100 (“data needed”). Once the labels have been obtained, the relevant case data is selected from various data sources (see operation 328) and attached to the initial ticket (see operation 329). This enriched ticket is called a suggested ticket (i.e., the enhanced support ticket request 50). The suggested ticket is presented to the ticket sender for reviewing and submission to the ticket recipient. During reviewing, the user can add or remove data that has been attached automatically based on the ML algorithm.

Referring back to FIG. 5, at an operation 330, the ticket is reviewed and submitted to the ticket recipient. At an operation 340, the ticket recipient processes the ticket. At an operation 350, both the initial ticket and results of processing the ticket are used to train the ML algorithm.

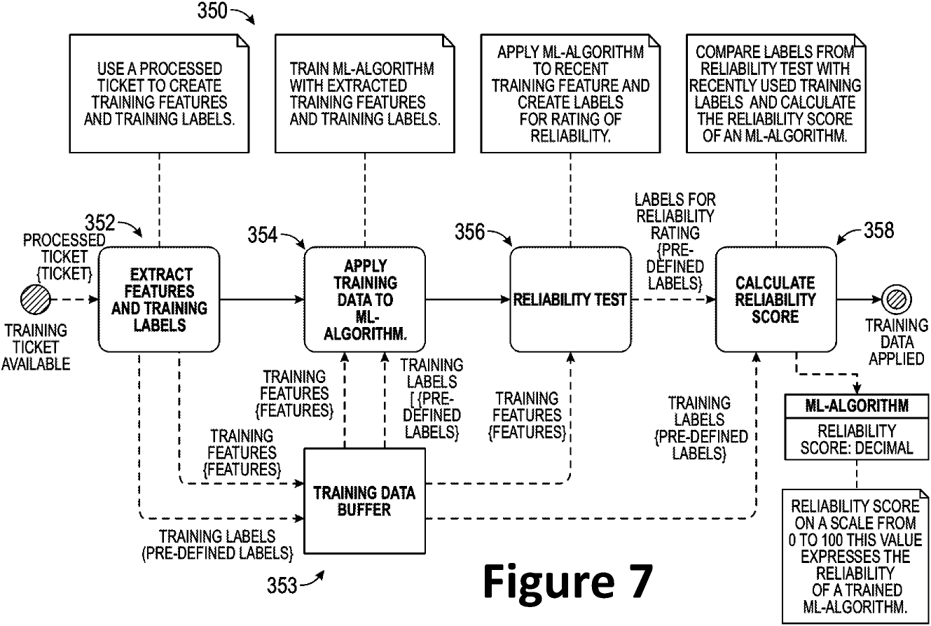

FIG. 7 shows a detailed example of the operation 350. When the ticket recipient processes the ticket, the recipient might use the attached data, but may also use any other missing data for correct processing. This data is automatically attached to the submitted ticket thus creating a potential training ticket. Any attached data that is not used for processing is removed from the training ticket. If the recipient regards the ticket as usable for training, then the recipient sets a marker, called “IsForTraining” to True. After the processing has been completed, the system stores the processed ticket in a buffer and evaluates the “IsForTraining” marker. If the marker has the value True, training of the MI,-Algorithm is invoked (Train MI,-Algorithm).

The processed ticket is used to extract training features and training labels (see operation 352) which are stored in a buffer (see operation 353). At an operation 354, the training data is feed applied the ML algorithm for learning. After the learning has been completed; a reliability test is performed with the features that were recently used for training (see operation 356). For this operation, the ML-algorithm is put into a production mode. The resulting labels are compared with the training labels that provide the ground truth (see operation 358), and a score is calculated that expresses the current reliability of the ML algorithm. The score is assigned to the algorithm for use during the production phase.

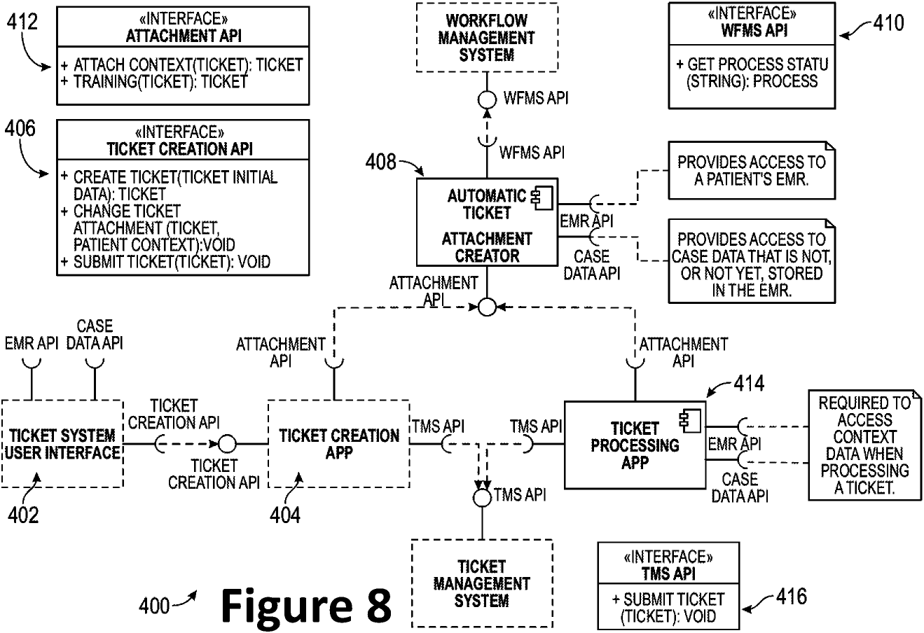

FIG. 8 shows another illustrative example of an automated ticketing system 400 for implementation in an existing IT environment. A Ticket System User Interface 402 and a Ticket Creation App 404 together create an initial ticket via a Ticket Creation Application Programming Interface (API) 406. An Automatic Ticket Attachment Creator 408 implements the ML algorithm that accesses a WIMS API 410, an EMR, and Case Data through APIs. Its functions are accessible through an Attachment API 412. The completed ticket is passed back to the Ticket System User Interface 402 for review by the users. The reviewed ticket is returned to the Ticket Creator App 404 and passed on for submission.

A Ticket Processing App 414 implements the handling of the ticket by the recipient. In one embodiment, the relevance values used for training are determined by the order of items in the reviewed ticket and/or the order of usage of these items by the recipient API for passing a processed ticket on for training via a ticket management system API 416.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

In the foregoing detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials, and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. Additionally, the terms “comprises,” “comprising,” and/or similar terms specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

1. A non-transitory computer readable medium storing instructions executable by at least one electronic processor to perform an automated ticketing method comprising:
receiving a support request ticket for assistance with a medical device, the support request ticket being entered by a requestor via a ticket entry user interface (UI) of an automated ticket management system;
analyzing the support request ticket to identify ticket enhancement information not included in the support request ticket;
retrieving the ticket enhancement information from one or more databases;
adding the ticket enhancement information to the support request ticket to generate an enhanced support request ticket; and
transmitting one of the support request ticket or the enhanced support request ticket to a ticket recipient electronic processing device operable by a ticket recipient.

2. The non-transitory computer readable medium of claim 1, wherein the method further includes:
displaying the enhanced support request ticket via the ticket entry UI; and
receiving, from the requestor, via the ticket entry US, a user input indicative of one of an acceptance of the enhanced support request ticket or a rejection of the enhanced support request ticket;
wherein the enhanced support request ticket is transmitted to the ticket recipient electronic processing device in response to receiving an acceptance of the enhanced support request ticket; and
wherein the support request ticket is transmitted to the ticket recipient electronic processing device in response to receiving a rejection of the enhanced support request ticket.

3. The non-transitory computer readable medium of claim 1, wherein analyzing the support request ticket includes:
inputting the support request ticket to a machine-learning (ML) component to identify the ticket enhancement information.

4. The non-transitory computer readable medium of claim 1, wherein the method further includes:
transmitting the enhanced support request ticket to the ticket recipient electronic processing device operable by a ticket recipient; and
establishing a natural language communication pathway between the ticket recipient electronic processing device and an electronic processing device operable by the ticket sender.

5. The non-transitory computer readable medium of claim 1, wherein the ticket enhancement information is determined by an imaging protocol of a medical imaging device used in a medical imaging procedure that is a subject of the support request ticket.

6. The non-transitory computer readable medium of claim 1, wherein the ticket enhancement information is determined by a clinical pathway of a medical imaging procedure that is a subject of the support request ticket.

7. The non-transitory computer readable medium of claim 1, wherein the ticket enhancement information is determined by a status of a medical imaging device used in a medical imaging procedure that is a subject of the support request ticket.

8. The non-transitory computer readable medium of claim 1, wherein the ticket enhancement information is determined by information about a sender of the support request ticket, wherein such sender information includes at least one of experience level, job description, general training experience, and specific training experience on a particular imaging protocol, an imaging modality, an imaging brand, or a clinical pathway of a medical imaging procedure.

9. The non-transitory computer readable medium of claim 1, wherein the ticket enhancement information is determined by information about a sender of the support request ticket, wherein such sender information includes a plurality of experience level, job description, general training experience, and specific training experience on a particular imaging protocol, an imaging modality, an imaging brand, or a clinical pathway of a medical imaging procedure.

10. The non-transitory computer readable medium of claim 1, wherein the method further includes:
at the ticket recipient electronic processing device, tracking other information that is not included in the ticket enhancement information that is used by the ticket recipient to assist with the medical device; and
updating training of the ML component based on the other information.

11. The non-transitory computer readable medium of claim 1, wherein the method further includes:
assigning a reliability score for the enhanced support request ticket.

12. A non-transitory computer readable medium storing instructions executable by at least one electronic processor to perform an automated ticketing method between an operator (LO) of a medical device and a ticket recipient (RE) being requested to assist the operator, the method comprising:
analyzing a support request ticket to identify ticket enhancement information not included in the support request ticket, the support request ticket including a request for assistance with a medical device and being entered by a requestor via a ticket entry user interface (UI) of an automated ticket management system;
retrieving the ticket enhancement information from one or more databases;
adding the ticket enhancement information to the support request ticket to generate an enhanced support request ticket;
transmitting one of the support request ticket or the enhanced support request ticket to a ticket recipient electronic processing device operable by a ticket recipient;
displaying the enhanced support request ticket via the ticket entry UI; and
receiving, from the requestor, via the ticket entry UI, a user input indicative of one of an acceptance of the enhanced support request ticket or a rejection of the enhanced support request ticket.

13. The non-transitory computer readable medium of claim 12, wherein:
the enhanced support request ticket is transmitted to the ticket recipient electronic processing device in response to receiving an acceptance of the enhanced support request ticket; and
the support request ticket is transmitted to the ticket recipient electronic processing device in response to receiving a rejection of the enhanced support request ticket.

14. The non-transitory computer readable medium of claim 12, wherein analyzing the support request ticket includes:
inputting the support request ticket to a machine-learning (ML) component to identify the ticket enhancement information.

15. The non-transitory computer readable medium of claim 12, wherein the method further includes:
transmitting the enhanced support request ticket to the ticket recipient electronic processing device operable by a ticket recipient; and
establishing a natural language communication pathway between the ticket recipient electronic processing device and an electronic processing device operable by the ticket sender.

16. The non-transitory computer readable medium of claim 12, wherein the ticket enhancement information is determined by one of an imaging protocol of a medical imaging device used in a medical imaging procedure that is a subject of the support request ticket, a clinical pathway of a medical imaging procedure that is a subject of the support request ticket, and a status of a medical imaging device used in a medical imaging procedure that is a subject of the support request ticket.

17. The non-transitory computer readable medium of claim 12, wherein the ticket enhancement information is determined by information about a sender of the support request ticket, wherein such sender information includes a plurality of experience level, job description, general training experience, and specific training experience on a particular imaging protocol, an imaging modality, an imaging brand, or a clinical pathway of a medical imaging procedure.

18. The non-transitory computer readable medium of claim 10, wherein the method further includes:
at the ticket recipient electronic processing device, tracking other information that is not included in the ticket enhancement information that is used by the ticket recipient to assist with the medical device; and
updating training of the ML component based on the other information.

19. The non-transitory computer readable medium of claim 10, wherein the method further includes:
assigning a reliability score for the enhanced support request ticket.

20. An automated ticketing method between a local operator (LO) performing a servicing session for a medical device and a remote expert (RE) assisting the local operator, the method comprising:
analyzing a ticket for assistance submitted by the local operator to the remote expert with a machine learning (ML) component to identify ticket enhancement information not included in the ticket;
retrieving the ticket enhancement information from one or more databases;
filling in one or more fields of the ticket with the retrieved ticket enhancement information to generate an enhanced ticket; and
transmitting the enhanced ticket to an electronic processing device operable by the remote expert.
